(19) United States
(12) Patent Application Publication
(10) Pub. No.: US 2003/0161828 A1
(43) Pub. Date: Aug. 28, 2003

(54) Use of TNF antagonists as drugs for the treatment of patients with an inflammatory reaction and without suffering from total organ failure

(75) Inventors: Heidrun Dorothea Abdelghany, Mannheim (DE); William T. Barchuk, Madison, NJ (US); Lothar Daum, Otterstadt (DE); Jurgen Eiselstein, Weisenheim am Berg (DE); Martin Kaul, Neustadt (DE); Lori L. Van Meter, Hackettstown, NJ (US)

Correspondence Address: LAHIVE & COCKFIELD, 28 STATE STREET, BOSTON, MA, 02109, US

(73) Assignee: Abbott Gmbh & Co. KG, Weisbaden (DE)

(21) Appl. No.: 10/079,776

(22) Filed: Feb. 19, 2002

(51) Int. Cl. A61K 39/395

(52) U.S. Cl. 424/145.1

(57) ABSTRACT

The present invention relates to the use of Tumor Necrosis Factor (TNF) antagonists in the treatment of patients with an inflammatory reaction and without suffering from total organ failure.

### RELATED APPLICATIONS

This application is related to application Ser. No. 60/xxx,xxx, filed on Feb. 19, 2002 entitled The Use of TNF Antagonists as Drugs for the Treatment of Patients With an Inflammatory Reaction and Without Suffering from Total Organ Failure.

### FIELD OF THE INVENTION

The present invention relates to the use of Tumor Necrosis Factor (TNF) antagonists in the treatment of patients with an inflammatory reaction, as for example sepsis, and without suffering from total organ failure.

### BACKGROUND OF THE INVENTION

It is known that the term tumor necrosis factor (TNF) embraces two cytotoxic factors (TNF-α and TNF-β) which are mostly produced by activated lymphocytes and monocytes. TNF-α is considered to be an important mediator of, i.a. sepsis.

EP-A-0 260 010 describes, for example, anti-TNF antibodies which are said to be utilizable for disorders associated with an increased level of TNF in the blood, such as sepsis (septic shock), transplant rejection, allergies, autoimmune diseases, shock lung, coagulation disturbances or inflammatory bone diseases, to inactivate TNF.

Examples of disorders characterized by high serum levels of interleukin-6 in patients are the sequelae of transplantations, autoimmune diseases and, in particular, certain types of septicemia.

Sepsis is defined in medical textbooks as a collective clinical term for conditions in which bacterial pathogens, starting from a focus, enter the blood stream to induce a wide range of subjective and objective pathological manifestations. It is furthermore found that the clinical picture may vary widely depending on the type of pathogen, the reactivity of the body, the primary focus and the changes in organ involvement (Sturm et al. “Grundbegriffe der Inneren Medizin”, 13th edition, page 570, Gustav Fischer Verlag, Stuttgart, 1984).

A number of cytokines have been suggested to be involved in the complex pathophysiological process of septicemia. TNF in particular is ascribed with an important role in septic shock on the basis of data from animal experiments (Beutler et al., Science 229 (1985) 869-871).

This has eventually led to clinical studies of the treatment of septic patients with anti-TNF antibodies. In a multicenter phase II study on the treatment of severe septicemia with a murine monoclonal anti-TNF antibody, however, it was found that the overall population (80 patients) did not profit in terms of survival rate from the treatment with the antibody. Only the patients with high circulating TNF concentrations appeared to profit, in terms of probability of survival, from high-dose anti-TNF antibody administration (C. J. Fisher et al., Critical Care Medicine, vol. 21, No. 3, pages 318-327). Furthermore, reference is made in this study to a correlation of the plasma levels of TNF and IL-6. A substantial portion of the study population suffered from severe sepsis and showed multiple organ dysfunction at study entry. Patients with organ dysfunction thus were definitely not excluded from said study.

The part played by the cytokine interleukin-6 (IL-6) in septicemia is unclear and contradictory. High serum levels of IL-6 have been found in some septic patients (Hack et al., Blood 74 (1989) 1704-1710).

Waage describes a correlation between the concentrations of the cytokines IL-6 and IL-8 with the severity of the shock, although they had no effect, either alone or in combination with TNF, on the development of a shock syndrome in terms of mortality (Waage in “Tumor Necrosis Factors”, ed. B. Beutler, Raven Press, New York, 1992, pages 275-283).

Some scientists have ascribed a beneficial role to IL-6 in septic shock because IL-6 inhibits, in the form of negative feedback control, the LPS-induced TNF production (Libert et al. in “Tumor Necrosis Factor: Molecular and Cellular Biology and Clinical Relevance”, ed. W. Fiers, Karger, Basel, 1993, pages 126-131).

WO-A-95/20978 discloses the use of TNF-antagonists in the treatment of disorders characterized by an increased IL-6 level. 122 patients were enrolled in an clinical study and it was observed that mortality could be significantly reduced by specifically treating those patients with a TNF-antagonist which have an initial serum IL-6 level of ≧500 pg/ml, in particular≧1000 g/ml. Patients with total organ failure were not excluded from that study.

### SUMMARY OF THE INVENTION

It is an object of the present invention to provide a modified approach for the treatment of patients with an inflammatory reaction, as for example septic patients, with TNF antagonists. In particular, it is an object of the present invention to obtain a more significant reduction of mortality upon administration of TNF-antagonists to patients with an inflammatory reaction, as for example septic patients.

It was now found, surprisingly, that TNF antagonists can be used particularly successfully as drugs for the treatment of those patients suffering from an inflammatory reaction, as for example sepsis, which do not show a total or full organ failure in any organ system.

Excluding specifically those patients from treatment with a TNF antagonist surprisingly allows a more pronounced reduction of mortality, or, in other word, a more pronounced increase of the survival rate, of the treated patients, relative to placebo.

### BRIEF DESCRIPTION OF THE DRAWINGS

FIG. 1 shows the mortality rate differences (versus placebo) observed for anti-TNF-alpha antibody treated septic patients with or without organ failure.

FIG. 2 shows the mortality rate differences (versus placebo) observed for anti-TNF-alpha antibody treated septic patients with or without organ failure and additionally having an IL-6 serum level of≧500 pg/ml.

FIG. 3 shows the mortality rate differences (versus placebo) observed for anti-TNF-alpha antibody treated septic patients with or without organ failure additionally having an IL-6 serum level of≧1000 pg/ml.

FIG. 4 shows the mortality rate differences (versus placebo) observed for anti-TNF-alpha antibody treated septic patients with or without organ failure having an high (elevated) IL-6 serum level as detected by SEPTEST™.

### DETAILED DESCRIPTION OF THE INVENTION

Unless otherwise stated, the term “TNF” as used herein is to be regarded as synonym for “TNF-alpha”.

Non-limiting examples of an “inflammatory reaction” according to the present invention comprise at least one of the following disease states: sepsis (septic schock), severe trauma, pancreatitis, burns, transplantation and diseases associated therewith, major surgery and anaphylactic reactions.

The present invention is further illustrated for the disease state “sepsis”, treatment of which is particularly preferred according to the present invention.

A. Selection of the Preferred Sub-population of Septic Patients

According to a first preferred embodiment of the invention a method of treating a patient with sepsis is provided, which patient is not suffering from total organ failure of any (at least one) organ system, which method comprises: administering a therapeutically effective amount of at least one tumor necrosis factor (TNF) antagonist to said patient. Thus merely a well-defined sub-population of the entire population of septic patients is therapeutically treated.

The selection of said preferred sub-population of septic patients may be based on any test or scoring system allowing a skilled person to distinguish between patients without total organ failure and patients with total organ failure in any organ system. Presently, two well established scoring systems designated “SOFA” and “MOD” are preferably applied according to the present invention. However, the present invention shall not be limited to the treatment of septic patients without total organ failure, selected by means of said two scoring systems. Any other scoring system not specifically mentioned herein or any future scoring system allowing to exclude patients with total organ failure of one single organ system, in particular of the respiratory, the renal, the hepatic, the cardiovascular, the hematologic and the neurologic system, may be applied as well and shall be considered to fall within the scope of the present invention.

A further preferred embodiment of the invention thus encompasses a method as defined above, wherein said total organ failure is defined by a Sequential Organ Failure Assessment (SOFA) score item and/or Multiple Organ Dysfunction (MOD) score item of equal to four (=4) in any organ system selected from the respiratory system, the renal system (kidney), the hepatic system (liver), the cardiovascular system, the hematologic system (coagulation system) and the neurologic system (CNS). Corresponding definitions for individual SOFA and MOD scores are given in the subsequent Tables 1 and2.

TABLE 1
Definition of Sequential Organ Failure Assessment (SOFA) Score

| Organ system | 1 | 2 | 3 | 4 |
|---|---|---|---|---|
| Respiratory | | | | |
| PaO₂/FiO₂, | <400 | <300 | <200 | <100 |
| (mmHg) | | | | |
| Hematologic | | | | |
| Platelets/nl | <150 | <100 | <50 | <20 |
| Hepatic | | | | |
| Bilirubin, | 1.2-1.9 | 2.0-5.9 | 6.0-11.9 | >12.0 |
| mg/dl | (20-32) | (33-101) | (102-204) | (>204) |
| (μmol/l) | | | | |
| Cardiovascular | | | | |
| Hypotension | MAP²⁾ <70 | Dopamine ≦5¹⁾ | Dopamine >5 or | Dopamine >15 or |
| | mmHG | or dobutamine | epinephrine ≦0.1¹⁾ | epinephrine >0.1 |
| | | (any dose) | or | or |
| | | | Norepinephrine | norepinebrine > |
| | | | <0.1¹⁾ | 0.1 |
| Neurologic | | | | |
| Glasgow Coma | 13-14 | 10-12 | 6-9 | <6 |
| Score | | | | |
| Renal | | | | |
| Creatinine, | 1.2-1.9 | 2.0-3.4 | 3.5-4.9 | >5.0 |
| mg/dl | (110-170) | (171-299) | (300-440) | (>440) |
| (μmol/l) | | | <500 ml/day | <200 ml/day |
| urine output | | | | |

¹⁾μg/kg body weight × min
²⁾MAP = Mean Aterial Pressure

TABLE 2
Definition of multiple Organ Dysfunction (MOD) score

| Organ | | | | | |
|---|---|---|---|---|---|
| System | 0 | 1 | 2 | 3 | 4 |
| Respiratory | | | | | |
| PO₂/FiO₂ | >300 | 226-300 | 151-225 | 76-150 | ≦75 |
| (mmHg) | | | | | |
| Renal | | | | | |
| serum | ≦100 | 101-200 | 201-350 | 351-500 | >500 |
| creatinine | | | | | |
| (μmol/liter) | | | | | |
| Hepatic | | | | | |
| serum | ≦20 | 21-60 | 61-120 | 121-240 | >240 |
| bilirubin | | | | | |
| (μmol/l) | | | | | |
| Cardiovascular | | | | | |
| PAR¹⁾ | ≦10.0 | 10.1-15.0 | 15.1-20.0 | 20.1-30.0 | >30.0 |
| Hematologic | | | | | |
| platelets/nl | >120 | 81-120 | 51-80 | 21-50 | ≦20 |
| Neurologic | | | | | |
| Glasgow | 15 | 13-14 | 10-12 | 7-9 | ≦6 |
| Coma Score | | | | | |

¹⁾PAR (Pressure-Adjusted Heart Rate) = HR × RAP (Right Atrial Pressure)/mean BP

The SOFA and MOD scoring systems are further explained in the following documents which are herewith incorporated by reference.

MOD: Marshall J C, Cook D J, Christou N V, Bernard G R, Sprung C L, Sibbald W J; Multiple Organ Dysfunction Score: A reliable descriptor of a complex clinical outcome. Critical Care Medicine 23 (1995), 1638-1652.

SOFA: Vincent J L, Moreno R, Takala J , Willatts S, De Mendoca A, Bruining H, Reinhart C K, Suter P M, Theijs L G; The SOFA (Sepsis-related Organ Failure Assessment) score to describe organ dysfunction/failure. Intensive Care Medicine 22 (1996), 707-710.

According to a further preferred embodiment the method of the present invention is performed with patients additionally having a significantly high or elevated level, as for example serum level, of a naturally occurring indicator molecule or transmitter connected to an inflammatory reaction. Typical examples are cellular transmitters involved in signal transduction, in particular cell surface receptors and corresponding ligands, as well as precursors and fragments derived therefrom.

Non-limiting examples of “a naturally occurring indicator molecule connected to an inflammatory reaction” are: IL-1, IL-1 receptor, IL-6, IL-8, soluble TNF receptors, in particular derived from p55 and p75 TNF receptors, TNF alpha, IL- 10, IL-12, IL-13, IL-18, CRP (C-reactive protein), alpha 2 macroglobuline and fibrinogen.

Particularly preferred is treatment of patients with a significantly high or elevated serum level of interleukin-6 (IL-6) at the beginning of the treatment. The high or elevated serum level can be determined in different ways well-known in the art, quantitatively or semi-quantitatively.

An “high or elevated level” or “high or elevated serum level” of an above identified indicator, as for example IL-6, means a level which is elevated at least ten-fold compared with physiologically normal serum levels in healthy subjects.

The “normal” levels or serum levels of indicators like IL-6 may be below the detection limit. This is in particular the case for IL-6, the level of which may vary slightly depending on the assay system used. For IL-6 the maximum “normal” level is, however, about 20 pg/ml. Preferably, an “high or elevated serum level” of IL-6 according to the present invention is therefore at least 200 pg/ml.

The treatment of septicemia with TNF antagonists is particularly successful according to this invention, for example measured by a distinct reduction in mortality, when the septic patients who are treated (patients without total organ failure of one organ system) have IL-6 serum levels of 500 pg/ml or more at the start of treatment. Patients who have IL-6 serum levels above 1000 pg/ml profit particularly well from the treatment according to the invention.

Serum concentrations of IL-6 up to 20,000 times the levels in healthy subjects have been found in septic patients, as for example 1×10⁶ pg/ml serum.

The serum concentrations of IL-6 can be determined quantitatively by conventional detection methods such as RIA or ELISA. Examples of very suitable detection systems are the IL-6-ELISA supplied by Medgenix or by Boehringer Mannheim/Roche (Human IL-6 ELISA, Roche Molecular Biochemicals, Cat. No. 1 534 475)

The IL-6 concentration can also be determined quantitatively by an activity assay in which, for example, C-reactive protein is assayed. As an example of a semi-quantitative IL-6 assay system there may be mentioned the SEPTEST™ test kit by Abbott.

Thus, according to a further preferred embodiment patients having a serum level of interleukin-6 (IL-6) of ≧500 pg/ml, more preferably ≧1000 pg/ml, are treated with a TNF antagonist. Alternatively said patients may have an increased serum level of IL-6 as determined by the semi-quantitative IL-6 testkit SEPTEST™. Said SEPTEST™ IL-6 test kit identifies septic patients with serum IL-6 levels above 1,000 pg/ml, with a 91.8% sensitivity as determined by the human IL-6 ELISA from Boehringer Mannheim/Roche (see above).

B. The TNF Antagonist

TNF antagonists useful for the present invention are selected from any type of compound, in particular pharmaceutically acceptable compounds, showing TNF antagonizing activity. In particular, low molecular compounds or high molecular compounds, in particular high molecular biomolecules, are encompassed.

As examples there may be mentioned compounds interacting on the DNA and/or RNA level with TNF alpha biosynthesis, as for example antisense RNA molecules or transcription inhibitors like pentoxyphyllin.

As TNF antagonist for performing the treatment according to the present invention there may in particular be applied any biomolecule having binding activity for TNF-alpha, and preferably any biomolecule with binding specificity for TNF-alpha of human and optionally at least one other primate TNF-alpha, as for example chimpanzee. “TNF antagonists” according to the present invention thus comprise preferably TNF-alpha specific antagonists, in particular those with TNF-alpha neutralizing activity. The TNF-alpha neutralizing activity can easily be tested by determining the antagonist's influence on TNF-alpha induced cytotoxicity in vivo or in vitro. Suitable tests are for example disclosed in WO-A-97/29131.

Suitable TNF antagonists are anti-TNF antibodies, TNF receptors, TNF binding proteins or those TNF derivatives which still bind to TNF receptors but have no TNF activity. Preferably said antagonists are of human origin (isolated from a human source or recombinantly prepared in a suitable host cells system). Anti-TNF antibodies are particularly preferred for the use according to the invention.

Anti-TNF antibodies suitable for the use according to the invention are well known (for example EP-A-0 260 610, EP-A-0 351 789, EP-A-0 218 868). Both polyclonal and monoclonal antibodies can be used. Furthermore, TNF-binding antibody fragments such as Fab or F(ab′)₂ fragments or single-chain Fv fragments are also suitable. A particularly preferred monoclonal anti-hTNF-alpha antibody is described in EP-A-0 260 610, designated AM-195 or MAK-195 and is produced by a hybridoma cell line deposited with the ECACC under the accession number 87 050803.

Furthermore, chimeric (for example human constant region/mouse variable region), humanized or human anti-TNF antibodies or their TNF-binding fragments are also very suitable because these molecules ought not to cause any anti-mouse antigenicity in human patients. Suitable humanized anti-TNF antibodies are for example disclosed in WO-A-97/29131.

Suitable TNF-alpha binding proteins are preferably those derived from the natural, membrane bound cell surface receptor molecules for TNF-alpha. At least two different receptor molecules are presently known, i.e. p55 TNF-R and p75 TNF-R. From the latter one two different soluble forms can be found in human urine. The first one with an apparent molecular weight of about 42 kDa determined by SDS gel electrophoresis (corresponding to the extra-cellular domain of the receptor molecule), and the second one with an apparent molecular weight of about 30 kDa, derivable from the first one by means of proteolytic cleavage (Porteu et al., 1991, J. Bio. Chem, 18846). Corresponding soluble TNF receptor fragments and derivatives thereof are, for example, disclosed in EP-A-0 471 701 (BASF AG), EP-A-0 398 327 (Yeda), EP-A-0 422 339 (Synergen) and U.S. Pat No. 5,945,397 (Immunex). As specific example for a suitable derivative of the soluble 42 kDa TNF binding molecule there may be mentioned fusion molecules of said 42 kDa fragment with the constant region of an immunoglobulin molecule. Corresponding fusion proteins are presently commercialized under the trade name ENBREL™.

It is also possible to use mixtures of various anti-TNF antibodies or of anti-TNF antibodies and TNF receptor fragments as active substance.

According to a preferred embodiment the claimed method is thus performed with a TNF-antagonist selected from a monoclonal anti-TNF-antibodies or fragments thereof, in particular human or humanized monoclonal antibodies; or with a TNF-receptor or a soluble fragment or derivative thereof with TNF-receptor activity.

C. Pharmaceutical Compositions

The present invention also includes pharmaceutical compositions which, besides non-toxic, inert, pharmaceutically suitable vehicles, contain at least one of said anti-TNF antagonists, and processes for the production of these compositions.

The anti-TNF antagonists are formulated in the conventional way for biotechnologically produced active substances, as a rule as liquid formulation or lyophilisate (see, for example, Hagers Handbuch der pharmazeutischen Praxis, vol. 2, 5th edition, 1991, p. 720, ISBN 3-540-52459-2). The above-mentioned pharmaceutical compositions are produced in a conventional way by conventional methods, e.g. by mixing the active substance or substances with the vehicle or vehicles.

D. Methods of Treatment

Administration of the TNF antagonist can take place as brief intravenous infusion of single doses or as continuous long-term infusion of the daily dose over 4 to 24 hours.

In general, it has proven advantageous to administer the active substance or substances which are suitable for the use according to the invention in total amounts of about 0.1 to about 1000, preferably 0.1 to 10, mg/kg of body weight every 24 hours, where appropriate in the form of several individual doses or as continuous infusion and, where appropriate, over a therapy period of several days to achieve the desired results. A single dose preferably contains the active substance or substances in amounts of about 0.1 to about 10 mg/kg of body weight. However, it may be necessary to deviate from the stated dosages, specifically depending on the age and size of the patient to be treated and on the nature and severity of the fundamental disorder, the type of composition and of administration of the drug, and the period or interval over which administration takes place.

The invention is illustrated further in the following example referring to the treatment of septic patients. This example is provided to aid in the understanding of the inventions and are not to be construed as a limitation thereof.

### EXAMPLE

### Treatment of Septic Patients with a Murine Anti-TNF Antibody Fragment (F(ab′)₂).

a. The Test Procedure

Septic patients were treated with a murine anti-TNF antibody fragment (F(ab')₂) also designated MAK 195F (INN: Afelimomab).

A total of 2634 patients with severe septicemia were analyzed in a multicenter clinical study. In a first step the initial IL-6 serum level was determined by mean of a rapid semi-quantitative test (SEPTEST™). A positive test result indicated an IL-6 level above about 1000 pg/ml. Each of the two groups thus formed were randomized and divided into a treatment (MAK195F) and a placebo (control) group.

Moreover, all patients were given a standard therapy for septic patients.

Of the 2634 patients 998 showed a positive IL-6 test result, 1636 patients showed a negative or inconclusive SEPTEST™ result. Of said 998 patients 448 patients were assigned to the treatment group and 510 patients were assigned to the placebo group.

MAK 195F was administered in single doses of 1 mg/kg of body weight The therapy was administered as short-term infusion in nine doses at 8 hour intervals (ie. for three days).

The number of survivors was determined 28 days after the beginning of therapy. Retrospectively, the patients were examined as to whether they suffered at study entry from severe organ failure of one of the six essential organs or organ systems: the respiratory system, the renal system (kidney), the hepatic system (liver), the cardiovascular system, the hematologic system (coagulation system) and the neurologic system (CNS). Assessment of severe organ failure was performed on the basis of the corresponding standard evaluation systems: MOD (Multiple Organ Dysfunction) or SOFA (Sequential Organ Failure Assessment) scoring. Severe organ failure for one single organ corresponds to the maximum score of =4 for an individual organ system.

b. The Results

b1. Mortality observed for antibody treated septic patients with or without organ failure versus placebo independent of their IL-6 level

(1) Patients with organ failure based on MOD score (at least one score value of 4 in the assessment of the single organs)

Mortality

| | |
|---|---|
| TREATMENT | 47.1% |
| Placebo | 49.8% |
| Difference | 2.7% |

(2) Patients with organ failure based on SOFA score (at least one score value of 4 in the assessment of the single organs)

Mortality

| | |
|---|---|
| TREATMENT | 43.1% |
| Placebo | 46.3% |
| Difference | 3.2% |

(3) Patients without organ failure based on MOD score (no score value of 4 for each item which assess the single organs)

Mortality

| | |
|---|---|
| TREATMENT | 24.6% |
| Placebo | 28.4% |
| Difference | 3.8% |

(4) Patients without organ failure based on SOFA score (no score value of 4 for each item which assess the single organs)

Mortality

| | |
|---|---|
| TREATMENT | 21.7% |
| Placebo | 25.7% |
| Difference | 4.0% |

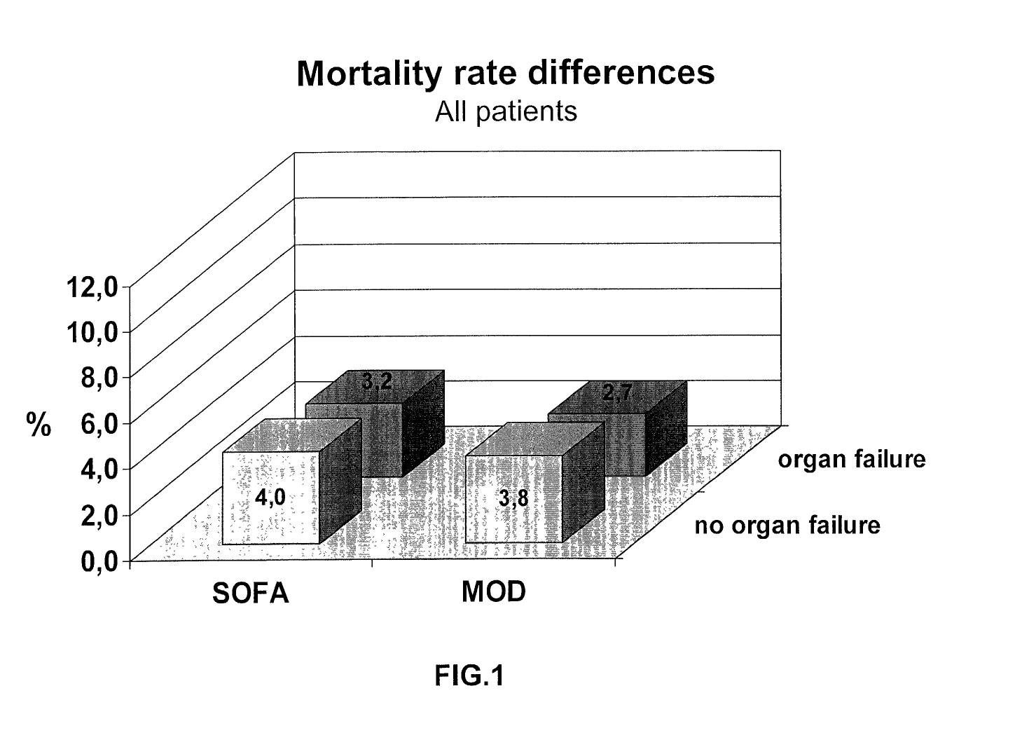

The results a summarized in FIG. 1.

b2) Mortality observed for antibody treated septic patients with or without organ failure and having an IL-6 serum level of ≧500 pg/ml

(1) Patients with organ failure based on MOD score (at least one score value of 4 in the assessment of the single organs)

Mortality

| | |
|---|---|
| TREATMENT | 54.5% |
| Placebo | 57.6% |
| Difference | 3.1% |

(2) Patients with organ failure based on SOFA score (at least one score value of 4 in the assessment of the single organs)

Mortality

| | |
|---|---|
| TREATMENT | 49.3% |
| Placebo | 54.4% |
| Difference | 5.1% |

(3) Patients without organ failure based on MOD score (no score value of 4 for each item which assess the single organs)

Mortality

| | |
|---|---|
| TREATMENT | 32.6% |
| Placebo | 39.7% |
| Difference | 7.1% |

(4) Patients without organ failure based on SOFA score (no score value of 4 for each item which assess the single organs)

Mortality

| | |
|---|---|
| TREATMENT | 30.0% |
| Placebo | 36.5% |
| Difference | 6.5% |

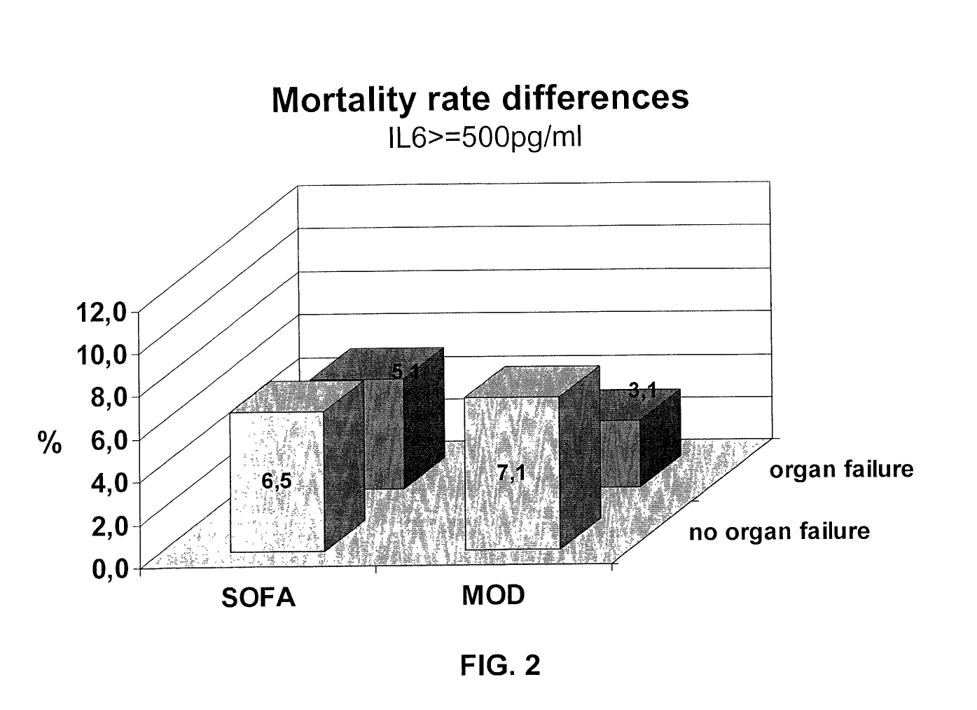

The results are summarized in FIG. 2.

b3) Mortality observed for antibody treated septic patients with or without organ failure and having an IL-6 serum level of ≧1000 pg/ml

(1) Patients with organ failure based on MOD score (at least one score value of 4 in the assessment of the single organs)

Mortality

| | |
|---|---|
| TREATMENT | 58.5% |
| Placebo | 58.9% |
| Difference | 0.1% |

(2) Patients with organ failure based on SOFA score (at least one score value of 4 in the assessment of the single organs)

Mortality

| | |
|---|---|
| TREATMENT | 52.9% |
| Placebo | 56.4% |
| Difference | 3.5% |

(3) Patients without organ failure based on MOD score (no score value of 4 for each item which assess the single organs)

Mortality

| | |
|---|---|
| TREATMENT | 34.3% |
| Placebo | 43.6% |
| Difference | 9.3% |

(4) Patients without organ failure based on SOFA score (no score value of 4 for each item which assess the single organs)

Mortality

| | |
|---|---|
| TREATMENT | 32.1% |
| Placebo | 40.2% |
| Difference | 8.1% |

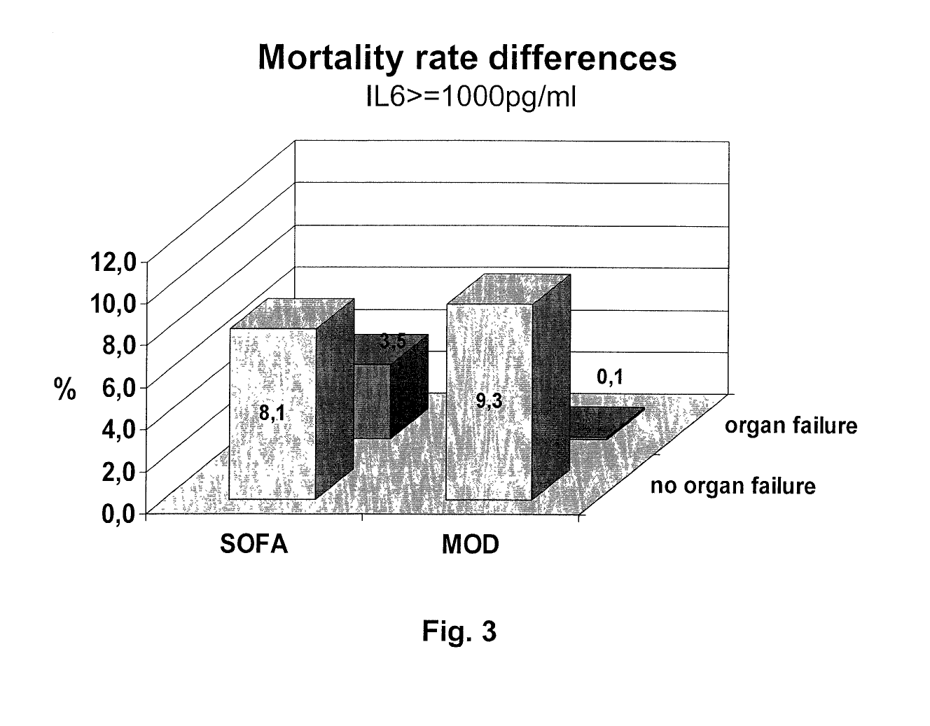

The results are summarized in FIG. 3.

b4) Mortality observed for antibody treated septic patients with or without organ failure and having a SEPTEST™ positive IL-6 serum level

(1) Patients with organ failure based on MOD score (at least one score value of 4 in the assessment of the single organs)

Mortality

| | |
|---|---|
| TREATMENT | 58.7% |
| Placebo | 59.8% |
| Difference | 1.1% |

(2) Patients with organ failure based on SOFA score (at least one score value of 4 in the assessment of the single organs)

Mortality

| | |
|---|---|
| TREATMENT | 53.5% |
| Placebo | 54.4% |
| Difference | 0.9% |

(3) Patients without organ failure based on MOD score (no score value of 4 for each item which assess the single organs)

Mortality

| | |
|---|---|
| TREATMENT | 30.8% |
| Placebo | 38.1% |
| Difference | 7.3% |

(4) Patients without organ failure based on SOFA score (no score value of 4 for each item which assess the single organs)

Mortality

| | |
|---|---|
| TREATMENT | 25.3% |
| Placebo | 36.3% |
| Difference | 11.0% |

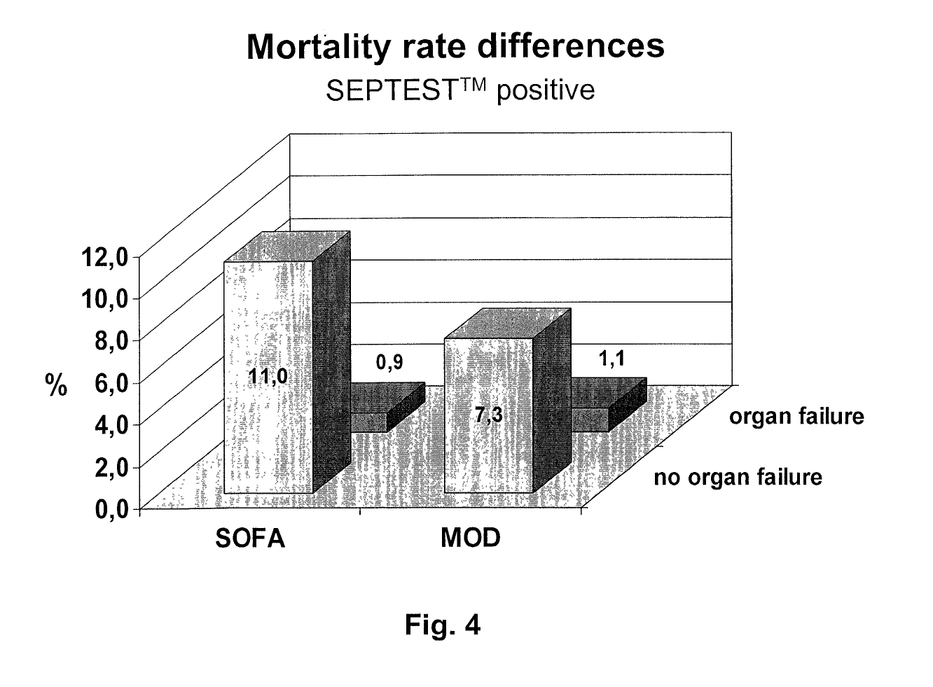

The results are summarized in FIG. 4.

The above results of this clinical study clearly prove that treatment of severe septicemia with anti-TNF antibodies is more successful if patients without total organ failure are treated, and, more particularly if patients are treated which additionally have a significantly high serum level of IL-6, as for example ≧500 or ≧1000 pg/ml.

Equivalents

Those skilled in the art will recognize, or be able to ascertain using no more than routine experimentation numerous equivalents to the specific procedures described herein. Such equivalents are considered to be within the scope of the present invention and are covered by the following claims. The contents of all references patents, and patent applications cited throughout this application are hereby incorporated by reference. The appropriate components, processes, and methods of those patents, application and other documents may be selected for the present invention as embodiments thereof.

1. A method of treating a patient with an inflammatory reaction which patient is not suffering from total organ failure of any organ system, which method comprises:
administering a therapeutically effective amount of a tumor necrosis factor (TNF) antagonist to said patient.

2. The method of claim 1, wherein said total organ failure is defined by a Sequential Organ Failure Assessment (SOFA) score item and/or Multiple Organ Dysfunction (MOD) score item of equal to 4 in at least one organ system selected from the respiratory system, the renal system, the hepatic system, the cardiovascular system, the hematologic/coagulation system and the neurologic system.

3. The method of claim 1 or 2, wherein said patient additionally has a significantly elevated level of a naturally occurring indicator molecule connected to an inflammatory reaction.

4. The method of claim 3, wherein said patient has a significantly high serum level of interleukin-6 (IL-6).

5. The method of claim 4, wherein said patient has a serum level of IL-6 of ≧500 pg/ml.

6. The method of claim 5, wherein said patient has a serum level of IL-6 of ≧1000 pg/ml.

7. The method of claim 4, wherein said patient has an increased serum level of IL-6 as determined by a semi-quantitative or qualitative IL-6 assay system.

8. The method of claim 1, wherein the TNF-antagonist is a monoclonal anti-TNF-antibody or a fragment thereof.

9. The method of claim 8, wherein said antibody is a human or humanized monoclonal antibody.

10. The method of claim 1, wherein said TNF-antagonist is a TNF-receptor or a soluble fragment or derivative thereof with TNF-receptor activity.

11. The method of claim 1, wherein patients with sepsis are treated.
